(19) United States
(12) Patent Application Publication
(10) Pub. No.: US 2003/0235814 A1
(43) Pub. Date: Dec. 25, 2003

(54) Compositions and methods for selecting open reading frames

(75) Inventors: Andrew R.M. Bradbury, Santa Fe, NM (US); Roberto Marzari, Trieste (IT)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Los Alamos National Laboratory, Los Alamos, NM (US)

(21) Appl. No.: 10/175,689

(22) Filed: Jun. 19, 2002

(51) Int. Cl. C12Q 1/70; C12N 7/00; C12N 15/74; C12N 1/21

(52) U.S. Cl. 435/5; 435/472; 435/235.1; 435/69.1; 435/252.3

(57) ABSTRACT

The current invention provides method and vectors for selecting open reading frames. Open reading frames present in a fragment of DNA cloned into the vectors of the invention result in creation of a fusion protein between the amino acid sequence encoded by the fusion protein and a reporter protein. The vector further comprises recombination sites so that once a recombinant that comprises an open reading frame is identified, either the reporter sequence or the open reading frame can be removed from the vector.

### STATEMENT AS TO RIGHTS TO INVENTIONS MADE UNDER FEDERALLY SPONSORED RESEARCH OR DEVELOPMENT

This invention was made with government support under grant number DE-FG02-98ER62647 awarded by the Department of Energy. The Government has certain rights in this invention.

### FIELD OF THE INVENTION

This invention provides compositions and methods for identifying and isolating open reading frames present in nucleic acid fragments.

### BACKGROUND OF THE INVENTION

Only approximately 1.5% of the human genome comprises functional open reading frames. One goal of the human genome project is to identify all human genes and the polypeptides encoded by the genes. Attempts to identify protein-coding regions in silico, using EST and whole genome sequence information have met with some success, however, true functional analysis of the activity of the products encoded by these genes requires the physical piece of DNA containing these genes. This problem has been addressed in C. elegans by using systematic amplification of the open reading frames of all predicted genes (see, e.g., Reboul et al. Nat Genet 27:332-336 (2001)), with evidence for at least 17,300 genes in this organism, of which a high proportion have structures different to those predicted in silico. The cloning of these open reading frames using a recombinatorial system (see, e.g., Hartley et al., Genome Res 10:1788-1795 (2000)) allows easy transfer to different vectors, and a similar strategy has been proposed for the human genome (see, e.g., Brizuela et al., Mol Biochem Parasitol 118:155-165 (2001)). This provides the potential means to generate complete collections of gene products, if high throughput methods to consistently produce proteins could be found. Such a complete collection potentially represents all the polypeptides expressed by an organism. Interrogation of the collection could be carried out in a protein chip format (see, e.g., Zhu et al., Scienc. 293:2101-2105 (2001)). However, this approach, apart from the considerable investment required, suffers from the problem that not all proteins can be easily expressed and purified.

An alternative method is to randomly fragment DNA enriched in coding sequences, and to rely on the variable expression of different polypeptides to provide overlapping fragmented representation of individual genes. This approach could be particularly useful in phage display, a technology originally developed to select peptide epitopes recognized by antibodies, but subsequently expanded to include the display of antibodies and many other proteins. Although phage display has been successfully applied to gene-rich bacterial genomes and individual genes to identify antibody epitopes or binding partners; the technique suffers from the problem that only one clone in eighteen, if starting with DNA encoding an open reading frame, will be correctly in frame (one clone in three will start correctly, one clone in three will end correctly, and one clone in two will have the correct orientation). While this high rate of non-functional inserts may be tolerable when starting with DNA from a single gene or even a small gene-rich genome, in which complete functional representation can be obtained with relatively small libraries, it becomes impractical if using more complex DNA sources.

In general, attempts to display random open reading frames on filamentous phage, such as those encoded by cDNA fragments have not been very successful, notwithstanding the development of vectors in which random fragments are displayed at the C terminus of a Jun peptide which interacts with Fos displayed at the N terminus of p3 (see, e.g. Crameri, et al., Eur. J. Biochem. 226:53-58 (1994); Crameri & Blaser, Int Arch Allergy Immunol 110:41-45 (1996)), at the C terminus of p3 (see, e.g., Fuh & Sidhu, FEBS Lett 480:231-234 (2000)), p8 (Fuh et al., J. Biol Chem 275:21486-21491 (2000)), p6 (Jespers et al, Biotechnology 13:378-382 (1995)), or at the C terminus of an artificial protein that is able to replace p8 in filamentous phage (Weiss & Sidhu, J. Mol. Biol. 300:213-219 (2000)). Greater success appears to have been achieved with lambda-based vectors for cDNA display (e.g., Santini et al., J. Mol. Biol. 282:125-135 (1998); Beghetto et al., Int J Parasitol 31:1659-1668 (2001)). However, even though these C terminal intracellular vectors increase the liklihood that open reading frames will be displayed, they do not per se, provide any selective pressure for open reading frames.

Thus, there is a need for a selective step to filter DNA fragments encoding open reading frames away from those which do not. The current invention addresses this need. Described herein are methods and compositions to select nucleic acid fragments encoding open reading frames from a library of sequences. In particular, the invention provides a system to identify open reading frames by positioning DNA fragments in an expression vector such that an open reading frame that is encoded by the fragment permits read-through to generate a fusion protein with a reporter, which provides a selectable phenotype.

Fusion proteins comprising an open reading frame fused to a selectable marker are known (see, e.g., Seehaus et al., Gene 114:235-237, 1992; and WO01/23602). However, these systems lack a means for removing the open reading frame from the selectable marker. The current invention provides a system in which the open reading frame identified using the screenable phenotype can be separated from the reporter molecule by recombination using recombination sites that are included in the vector and which flank either the reporter sequence or the open reading frame. This feature is useful for a variety of purposes. For example recombination mediated by sites flanking the open reading frame provides an efficient means of creating additional vectors, e.g., such as two-hybrid vectors, for functional analysis. Moreover, recombination mediated by sites flanking the reporter provide a mechanism for removing the reporter sequence from display vector systems, thereby creating an open reading frame-display protein fusion with no reporter sequences.

### BRIEF SUMMARY OF THE INVENTION

The current invention provides expression vectors and methods of using the vectors to identify and select an open reading frame encoded by a nucleic acid fragment.

In particular, the invention provides an expression vector comprising a cloning site upstream of a first nucleic acid sequence encoding a reporter protein, and a first and a second recombination site, wherein the first recombination site is situated between the cloning site and the first nucleic acid sequence encoding the reporter protein, and the second recombination site is situated between the first nucleic acid sequence encoding the reporter protein and the cloning site. This can be either downstream of the first nucleic acid sequence encoding the reporter protein or upstream of the cloning site. Expression of an open reading frame present in a nucleic acid fragment inserted into the cloning site results in a fusion protein comprising the sequence expressed from the open reading frame and the reporter protein. Typically, when recombination sites flank the selection marker, the sites are homologous; when they flank the selected open reading frame gene they are non-homologous, e.g., so that they can be transferred to another vector.

The expression vector can also comprises a second nucleic acid sequence encoding a polypeptide, wherein the nucleic acid encoding the polypeptide is in-frame with the homologous recombination sites, the reporter protein, and the open reading frame. In one embodiment, the polypeptide is a display protein, such as a virus coat protein. The display protein is often from a filamentous phage, e.g., fd phage, an fl phage, an M13 phage, an Ike phage, or any hybrids thereof; a lambda phage; or a T7 phage. In one embodiment, the coat protein is filamentous phage gene 3, filamentous phage gene 7 or filamentous phage gene 6.

The expression vectors of the invention can be expressed in any cell, such as a bacterial cell, a yeast cell, or another eukaryotic cell.

In one embodiment, the first and the second recombination site are lox sites.

The reporter protein can be any number of proteins that have a detectable phenotype. These include a protein, such as β-lactamase or chloramphenicol acetyltransferase, that is encoded by an antibiotic resistance gene. Other reporter proteins include proteins that produce a detectable signal or color, such as green fluorescent protein, or β-galalctosidasegene.

Any nucleic acid can be evaluated for the presence of an in-frame open reading frame using the vectors and methods of the invention. Preferably, the nucleic acid is a genomic DNA or cDNA fragment.

In another aspect, the invention provides a method of identifying an open reading frame in a nucleic acid fragment, the method comprising: expressing a population of nucleic acid fragments in an expression vector comprising: a cloning site upstream of at nucleic acid sequence encoding a reporter and a first and a second recombination site, wherein the first recombination site is situated between the cloning site and the nucleic acid sequence encoding the reporter protein, and the second recombination site is situated either downstream of the nucleic acid sequence encoding the reporter protein or upstream of the cloning site; wherein expression of an open reading frame present in a nucleic acid fragment inserted into the cloning site results in a fusion protein comprising the sequence expressed from the open reading frame and the reporter protein; and selecting the vector that expresses the fusion protein.

The method can further comprise a step of removing the reporter sequence by recombination or a step of removing the nucleic acid fragment encoding the open reading frame by recombination, which nucleic acid fragment can then be used in other vectors, e.g., two-hybrid vectors, for functional analysis.

In another aspect the invention provides a display library comprising members that express a population of nucleic acid fragments that encode open reading frames, wherein an open reading expressed by a member of the library is joined in frame to a framework display region, and further, wherein sequences that are encoded by a recombination site are present between the framework display region and the open reading frame.

In another embodiment, a display library of the invention comprises members that express a population of nucleic acid fragments that encode open reading frames, wherein an open reading present in a nucleic acid fragment is expressed as a component of a fusion protein, wherein the fusion protein further comprises a reporter protein and a display protein.

### BRIEF DESCRIPTION OF THE DRAWINGS

FIG. 1 depicts an example of a method of selecting open reading frames using β-lactamase as a reporter protein. Random DNA fragments are cloned upstream of a β-lactamase gene, which confers ampicillin resistance. Those fragments that are open reading frames permit read through into the β-lactamase gene and confer ampicillin resistance. Those that are out of frame, or contain stop codons, do not survive. After selection on ampicillin, the β-lactamase gene can be removed by passage through bacteria expressing cre recombinase. The selected open reading frame can then be displayed on phage.

FIG. 2a provides a map of plasmid pPAO2. FIG. 2b depicts the polylinker sequence between the HindIII and EcoRI site of pUC119. The amino acid sequence of the in-frame construct is provided above the DNA sequences. “FS” indicates a frameshift, which is only overcome if an in-frame DNA fragment is present in the cloning site. FIG. 2C is a schematic of the LIC cloning procedure.

FIG. 3 provides exemplary results showing that only clones with open reading frames survive selection on ampicillin. D1.3 scFv, or an out-of-frame derivative, was cloned into pPAO2 and bacteria were plated on different concentrations of chloramphenicol or ampicillin with or without 1% glucose. Chloramphenicol resistance is encoded by the backbone of the plasmid, while ampicillin resistance is generated by the presence of in-frame fusions. Clone survival on ampicillin is expressed as a percentage of the number of clones growing on chloramphenicol.

FIG. 4 depicts the results of an experiment showing that ampicillin selection generates randomly sized open reading frames. FIG. 4a: 20 random clones taken from a plasmid generated fragment library, after selection on ampicillin and passage through a cre-expressing strain to remove β-lactamase, were amplified with primers spanning the cloning site. All fragments are different sizes, indicating that different random fragments were cloned. FIG. 4b: clones from FIG. 4a were digested with BstnI. All clones show different fragment sizes. FIG. 4c: 96 random clones from the same library were dotted onto nitrocellulose after induction of protein fragment expression with IPTG. The results show that about 80% of these clones have signal, indicating that an open reading frame was selected.

FIG. 5 shows the results of a sequence analysis of ampicillin-selected open reading frame clones. The plasmid pET28b-tTG is represented linearly, with all open reading frames over 150 base pairs (bps) indicated. Open reading frames for kanamycin, tTG and rop (a protein involved in plasmid copy number control (Cesareni, et al, Proc Natl Acad Sci USA 79:6313-6317, 1982)) are indicated. Forty three sequences obtained from the library after selection on ampicillin and removal of the β-lactamase after passage through a cre-expressing strain are shown as lines beneath the appropriate frame.

FIG. 6 shows the identification of kanamycin clones. Three kanamycin specific primers were used in two separate PCR reactions with different numbers of estimated clones from the library indicated. Amplification was noted when 100 templates were present, indicating that the kanamycin sequence is present at an approximate level of 1/100. The 15-13 primers amplify and ORF of no biological significance, and is not represented in the library. The bars on the left indicate the length of the amplification products.

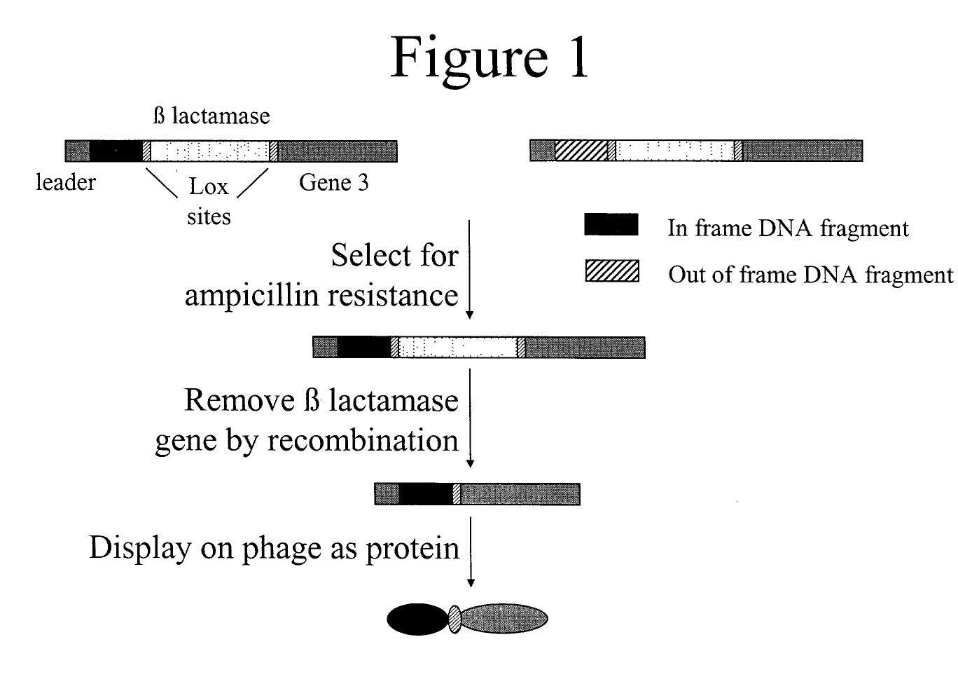

### DETAILED DESCRIPTION OF THE INVENTION

### Introduction

The current invention provides vectors and methods for filtering out open reading frames from a population of nucleic acid fragments. By cloning random nucleic acid fragments upstream of a gene for a reporter protein to create a fusion protein, only those clones containing DNA fragments encoding open reading frames permit expression of the reporter protein, which confers a detectable phenotype. The reporter protein or the open reading frame can then be removed via recombination using recombination sites that are also included in the vector.

The recombination sites can be situated in any place to separate the open reading frame from the reporter. Therefore, one of the recombination sites is positioned between the cloning site for the DNA fragment and the sequence encoding the reporter protein. A second recombination site is positioned upstream of the cloning site or downstream of the reporter protein. Thus, recombination will result in excision of the DNA fragment present in the cloning site, or excision of the reporter protein, respectively. In the former case, the open reading frame can then be inserted into another vector.

A vector of the invention can also comprise an additional nucleic acid encoding another protein that is in frame with and downstream of the reporter sequence. The open reading frame thus results in a fusion protein comprising the polypeptide encoded by the open reading frame, the reporter, and the additional polypeptide. The reporter sequence can then be removed, thereby resulting in a fusion comprising the open reading frame and the additional polypeptide. For example, the additional polypeptide can be a display protein. A display protein provides a framework to display the protein sequence encoded by the open reading frame on the surface of a particle. Thus, removal of the reporter protein can be important for presentation of the open reading frame. The display protein is often a virus protein, but can also be from other procaryotic and eukaryotic sources, such as a yeast or E. coli proteins.

### Definitions

The term “recombination site” or “site-specific recombination site” refers to a sequence of bases in a nucleic acid molecule that is recognized by a recombinase (along with associated proteins, in some cases) that mediates exchange or excision of the nucleic acid segments flanking the recombination sites. The recombinases and associated proteins are collectively referred to as “recombination proteins” (see, e.g., Landy, A., Current Opinion in Biotech. 3:699-707; 1993).

Numerous recombination systems from various organisms have been described (see, e.g., Hoess, et al., Nucl. Acids Res. 14:2287, 1986; Abremski, et al., J Biol. Chem. 261:391, 1986; Campbell, J. Bacteriol. 174:7495, 1992; Qian, et al., J. Biol. Chem. 267:7794; 1992; Araki, et al., J. Mol. Biol. 225:25, 1992; Maeser and Kahnmann, Mol. Gen. Genet. 230:170-176, 1991; Esposito, et al, Nucl. Acids Res. 25:3605, 1997.) Many of these belong to the integrase family of recombinases (Argos, et al., EMBO J 5:433-440 (1986); Voziyanov, et al., Nucl. Acids Res. 27:930, 1999). These include the Integrase/att system from bacteriophage .lambda.(Landy, A. Current Opinions in Genetics and Devel. 3:699-707, 1993)), the Cre/loxP system from bacteriophage P1 (Hoess & Abremski In Nucleic Acids and Molecular Biology, vol. 4. Eds.: Eckstein and Lilley, Berlin-Heidelberg: Springer-Verlag; pp.90-109, 1990), and the FLP/FRT system from the Saccharomyces cerevisiae 2 mu circle plasmid (Broach, et al., Cell 29:227-234, 1982).

A “recombination substrate” refers to a nucleic acid molecule that can recombine with another recombination substrate, either through the presence of specific recombination sites, e.g., loxP) or through similarities in the nucleic acid sequences that allow general cellular homologous recombination to occur.

The term “loxP site” refers to a recombinase recognition site that is a recombinase recognition site for Cre. A loxP site includes native lox sequences as well as modified loxP sites. Modified loxP sites are well known to those of skill in the art and include, but are not limited to, loxP511, fas, 2372, and other various mutations such as have been described n the literature.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another (see, e.g., Creighton, Proteins (1984)):

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

“Operably linked” as used herein means that the transcriptional and translational regulatory nucleic acid is positioned relative to any coding sequences in such a manner that transcription is initiated. Generally, this will mean that the promoter and transcriptional initiation or start sequences are positioned 5′ to the coding region. The transcriptional and translational regulatory nucleic acid will generally be appropriate to the host cell used, as will be appreciated by those in the art.

“Antibody” refers to a polypeptide substantially encoded by an immunoglobulin gene or immunoglobulin genes, or fragments thereof which specifically bind and recognize an antigen. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially an Fab with part of the hinge region (see, FUNDAMENTAL IMMUNOLOGY, 3D ED., Paul (ed.) 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies or those synthesized de novo using recombinant DNA methodologies (e.g., single chain Fv).

The phrase “single chain Fv” or “scFv” refers to an antibody in which the heavy chain and the light chain of a traditional two chain antibody have been joined to form one chain. Typically, a linker peptide is inserted between the two chains to allow for proper folding and creation of an active binding site.

The phrase “specifically (or selectively) binds” to a binding partner, e.g., an antigen, or “specifically (or selectively) reactive with,” when referring to a protein or peptide, refers to a binding reaction that is determinative of the presence of the protein in a heterogeneous population of proteins and other biologics. Thus, under designated assay conditions, a member of a binding pair binds to a particular protein above background, e.g., at least two times the background, and does not substantially bind in a significant amount to other proteins present in the sample. Typically a specific or selective reaction will be at least twice background signal or noise and more typically more than 1.0 to 100 times background.

A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

“Domain” refers to a unit of a protein or protein complex, comprising a polypeptide subsequence, a complete polypeptide sequence, or a plurality of polypeptide sequences where that unit has a defined function. The function is understood to be broadly defined and can be binding to a binding partner, catalytic activity or can have a stabilizing effect on the structure of the protein.

“Link” or “join” refers to any method of functionally connecting peptides, including, without limitation, recombinant fusion, covalent bonding, disulfide bonding, ionic bonding, hydrogen bonding, and electrostatic bonding. In the systems of the invention, a sequence encoding an open reading frame is typically joined, using recombinant DNA techniques, at its C-terminus to a reporter molecule. The reporter molecule can be a complete polypeptide, or a fragment or subsequence thereof. The polypeptide encoded by an open reading frame is typically indirectly linked to the reporter, e.g., via a linker sequence.

A “linker sequence” refers to an amino acid sequence that joins two heterologous polypeptides, or fragments or domains thereof. In general, as used herein, a linker is an amino acid sequence that covalently links the polypeptides to form a fusion polypeptide. A linker typically includes the amino acids translated from the remaining recombination signal after removal of a reporter gene from a display vector to create a fusion protein comprising an amino acid sequence encoded by an open reading frame and the display protein. As appreciated by one of skill in the art, the linker can comprise additional amino acids, such as glycine and other small neutral amino acids (e.g., [Gly-Gly-Gly-Gly-Ser]x).

“Fused” refers to linkage by covalent bonding.

A “fusion protein” refers to a protein comprising at least one polypeptide or fragment or domain thereof, that is linked or joined to a second polypeptide, or fragment or domain thereof.

A “reporter” protein refers to a protein that has a detectable activity that can be used to identify a cell that expresses the reporter protein. The term “reporter protein” also includes reference to fragments of the protein that have reporter activity.

The term “display protein” refers to a protein at least part of which is expressed on the surface of a particle, e.g., a virus or cell. The “display protein” can be encoded by nucleic acid native to the virus or cell, or can be encoded by a heterologous nucleic acid. A “display protein” serves as a vehicle for presenting a polypeptide sequence that is present in a fusion protein comprising the display protein and a polypeptide encoded by a heterologous nucleic at the surface of a particle.

A “cloning site” is a position in a vector to introduced a heterologous nucleic acid into a vector. Typically, the “cloning site” is the site of introduction of a DNA fragment to be analyzed for the presence of an open reading frame. Thus, in the current invention, the cloning site is positioned upstream of a nucleic acid sequence encoding a reporter such that an open reading frame in the DNA fragment leads to the creation of a fusion protein comprising the protein sequence encoded by the open reading frame and the reporter protein.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated nucleic acid sequences arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

As used herein, “isolate,” when referring to a molecule or composition, such as, for example, a polypeptide or nucleic acid or phage, means that the molecule or composition is separated from at least one other compound, such as a protein, other nucleic acids (e.g., RNAs), or other contaminants with which it is associated in vivo or in its naturally occurring state. Thus, a nucleic acid or phage is considered isolated when it has been isolated from any other component with which it is naturally associated, e.g., cell membrane, as in a cell extract. An isolated composition can, however, also be substantially pure. An isolated composition can be in a homogeneous state and can be in a dry or an aqueous solution. Purity and homogeneity can be determined, for example, using analytical chemistry techniques such as polyacrylamide gel electrophoresis (SDS-PAGE) or high performance liquid chromatography (HPLC).

The term “nucleic acid” or “nucleic acid sequence” refers to a deoxyribonucleotide or ribonucleotide oligonucleotide in either single- or double-stranded form. The term encompasses nucleic acids, i.e., oligonucleotides, containing known analogues of natural nucleotides which have similar or improved binding properties, for the purposes desired, as the reference nucleic acid. The term also includes nucleic acids which are metabolized in a manner similar to naturally occurring nucleotides or at rates that are improved thereover for the purposes desired. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see, e.g., Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., in WO 97/03211; WO 96/39154; Mata (1997) Toxicol. Appl. Pharmacol. 144:189-197. Other synthetic backbones encompasses by the term include methyl-phosphonate linkages or alternating methylphosphonate and phosphodiester linkages (Strauss-Soukup (1997) Biochemistry 36:8692-8698), and benzylphosphonate linkages (Samstag (1996) Antisense Nucleic Acid Drug Dev. 6:153-156). The term nucleic acid is used interchangeably with gene, cDNA, mRNA, oligonucleotide primer, probe and amplification product.

A “display vector” refers to a vector used to create a cell or virus that displays, i.e., expresses a display protein comprising a heterologous polypeptide, on its surface or in a cell compartment such that the polypeptide is accessible to test binding to target molecules of interest, such as antigens.

A “display library” refers to a population of display vehicles, often, but not always, cells or viruses. The “display vehicle” provides both the nucleic acid encoding a peptide as well as the peptide, such that the peptide is available for binding to a target molecule and further, provides a link between the peptide and the nucleic acid sequence that encodes the peptide. Various “display libraries” are known to those of skill in the art and include libraries such as phage, phagemids, yeast and other eukaryotic cells, bacterial display libraries, plasmid display libraries as well as in vitro libraries that do not require cells, for example ribosome display libraries or mRNA display libraries, where a physical linkage occurs between the mRNA or cDNA nucleic acid, and the protein encoded by the mRNA or cDNA.

A “phage expression vector” or “phagemid” refers to any phage-based recombinant expression system for the purpose of expressing a nucleic acid sequence in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

A “phage display library” refers to a “library” of bacteriophages on whose surface is expressed exogenous peptides or proteins. The foreign peptides or polypeptides are displayed on the phage capsid outer surface. The foreign peptide can be displayed as recombinant fusion proteins incorporated as part of a phage coat protein, as recombinant fusion proteins that are not normally phage coat proteins, but which are able to become incorporated into the capsid outer surface, or as proteins or peptides that become linked, covalently or not, to such proteins. This is accomplished by inserting an exogenous nucleic acid sequence into a nucleic acid that can be packaged into phage particles. Such exogenous nucleic acid sequences may be inserted, for example, into the coding sequence of a phage coat protein gene. If the foreign sequence is “in phase” the protein it encodes will be expressed as part of the coat protein. Thus, libraries of nucleic acid sequences, such as a genomic library from a specific cell or chromosome, can be so inserted into phages to create “phage libraries.” As peptides and proteins representative of those encoded for by the nucleic acid library are displayed by the phage, a “peptide-display library” is generated. While a variety of bacteriophages are used in such library constructions, typically, filamentous phage are used (Dunn (1996) Curr. Opin. Biotechnol. 7:547-553). See, e.g., description of phage display libraries, below.

The term “enrich for open reading frames” refers increasing the proportion of open reading frames present in a library comprising a vector into which nucleic acid fragments comprising candidate open reading frames have been inserted.

A “component of a protein binding detection system” refers to a polypeptide that is used in a system to detect binding between binding pair members. Such systems include display systems, e.g., phage display libraries, and two hybrid systems.

Nucleic Acid Sequences Comprising Open Reading Frames

Nucleic acids encoding the nucleic acid sequences to be expressed in the systems of the invention can be obtained using routine techniques in the field of recombinant genetics. Basic texts disclosing the general methods of use in this invention include Sambrook and Russell, MOLECULAR CLONING, A LABORATORY MANUAL (3rd ed. 2001) and CURRENT PROTOCOLS IN MOLECULAR BIOLOGY (Ausubel et al., eds., John Wiley & Sons, Inc. 1994-1997, 2001 version)).

Often, the nucleic acid sequences being evaluated for the presence of open reading frames are isolated using amplification techniques or obtained from fragmenting DNA.

Examples of techniques sufficient to direct persons of skill through in vitro amplification methods are found in Berger, Sambrook, and Ausubel, as well as Dieffenfach & Dveksler, PCR Primers: A Laboratory Manual (1995): Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117. These techniques are further discussed below.

### Fragmenting of Nucleic Acid Sequences

Often, the vectors for filtering out open reading frames comprises nucleic acids generated by fragmentation of either genomic DNA or cDNA. Methods for making genomic or cDNA libraries are also well known in the art, see e.g., Sambrook, Ausubel, Tijssen. In one exemplary means to make a library, genomic or cDNA, is extracted, purified and fragmented into subsequences. Fragmented nucleic acid of appropriate size is produced by known methods, such as nebulization, mechanical shearing or enzymatic digestion, to yield DNA fragments. Once the genomic DNA being analyzed has been fragment, the genomic nucleic acid fragments of desired size are then separated, e.g., by gradient centrifugation, or gel electrophoresis, from undesired sizes. The sizes of the fragments included in the desired population range can vary, depending on the vector, the size of the library, and the complexity of the sequences being analyzed. Often, the fragments are from about 20 base pairs to about 200 base pairs in size. However, smaller fragments, e.g., 10, or 15 base pairs; or larger fragments, e.g., 250, 300, 400, 500, 1,000 base pairs or longer, can also be used. The fragments are then inserted into the “filter” vectors for selection and identification of open reading frames. For phage libraries, the vectors and phage can be packaged in vitro or in vivo.

### Amplification of Nucleic Acids

Nucleic acids can also be generated for subcloning into a open reading frame selection vector using any amplification methodology known in the art using a variety of hybridization techniques and conditions. Amplification can be used for, e.g., the construction of hybridization probes or clones, identification, sequencing, quantification, and the like. Suitable amplification methods include, but are not limited to: polymerase chain reaction, PCR (PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y. (Innis )), ligase chain reaction (LCR) (Wu (1989) Genomics 4:560; Landegren (1988) Science 241:1077; Barringer (1990) Gene 89:117); transcription amplification (Kwoh (1989) Proc. Natl. Acad. Sci. USA 86:1173); and, self-sustained sequence replication (Guatelli (1990) Proc. Natl. Acad. Sci. USA, 87:1874); Q Beta replicase amplification and other RNA polymerase mediated techniques (e.g., NASBA, Cangene, Mississauga, Ontario); see Berger (1987) Methods Enzymol. 152:307-316, Sambrook, and Ausubel, as well as Mullis (1987) U.S. Pat. Nos. 4,683,195 and 4,683,202; Arnheim (1990) C&EN 36-47; Lomell J. Clin. Chem., 35:1826 (1989); Van Brunt, Biotechnology, 8:291-294 (1990); Wu (1989) Gene 4:560; Sooknanan (1995) Biotechnology 13:563-564. Methods for cloning in vitro amplified nucleic acids are described in Wallace, U.S. Pat. No. 5,426,039. Methods of amplifying large nucleic acids are summarized in, e.g., Cheng (1994) Nature 369:684-685.

For example, PCR can be used in a variety of protocols to amplify, identify, quantify, isolate and manipulate nucleic acids. In these protocols, primers and probes for amplification and hybridization are generated using known sequences or can be random sequences.

PCR-amplified sequences can also be labeled and used as detectable probes. The labeled amplified DNA or other oligonucleotide or nucleic acid of the invention can be used as probes to further identify and isolate, or identify and quantify, exons or antibody-encoding sequences from any source of nucleic acid, including, RNA, cDNA, genomic DNA, genomic libraries, in situ nucleic acid, and the like.

Recombination Systems

The reporter sequence or the open reading frame is removed from the vector by recombination. Recombination is mediated by specific recognition sequences, often termed “recombination sites,” on the nucleic acid molecules or subsequences of the nucleic acid molecule participating in the reactions. These recombination sites are sites that are recognized and bound by the recombination proteins during the initial stages of recombination.

The vectors of the invention typically comprise two recombination sites, one that is positioned between the cloning site into which the fragments with the candidate open reading frames are cloned, and the second, which is positioned either downstream of the nucleic acid sequence encoding the reporter protein or upstream of the cloning site. As appreciated by one of skill in the art, a recombination site that is between the cloning site and reporter protein must be in-frame to create a fusion protein between the open reading frame and the reporter protein. Similarly, if an additional polypeptide, such as a display protein, is also included in the construct, a recombination site that is situated between the candidate open reading frame and the nucleic acid sequence encoding the display framework region must be in-frame.

In many embodiments of the invention, it is desirable to remove the reporter sequence. Accordingly, the second recombination site is located downstream of the reporter sequence, often between the reporter sequence and an additional polypeptide sequence, e.g., a display protein. The second recombination site is usually homologous to the first site, such that recombination deletes the reporter sequence between the two sites. The recombination event typically leaves a residual recombination site subsequence, which is in-frame with the display protein. Thus, the fusion protein created after removal of the reporter protein will additionally comprise an amino acid sequence that is encoded by the residual recombination site sequences.

In another embodiment, the second recombination site is located upstream of the cloning site. In this embodiment, the recombination sites are often heterologous sites. Recombination can therefore be performed in which the nucleic acid sequence inserted in the cloning site is removed and then, for example, transferred to another plasmid via the heterologous recombination sites. Transfer is accomplished by passing the library through a host that expresses the appropriate recombinase system and that harbors the plasmid to which the sequence in the cloning site is to be transferred.

Numerous recombination systems from various organisms can be used in the invention. (see, e g., Hoess et al. (1986) Nucleic Acids Res. 14 (6): 2287; Abremski et al. (1986) J. Biol. Chem. 261 (1): 391; Campbell (1992) Bacteriol. 174 (23): 7495; Qian et al. (1992) J. Biol. Chem. 267 (11): 7794; Araki et al. (1992) J. Mol. Biol. 225 (1): 25). Many of these belong to the integrase family of recombinases (Argos et al. (1986) EMBO J. 5: 433-440), such as the Integrase/att system from bacteriophage λ (Landy (1993) Current Opinions in Genetics and Devel. 3: 699-707), the Cre/loxP system from bacteriophage P1 (Hoess and Abremski (1990) In Nucleic Acids and Molecular Biology, vol. 4, Eckstein and Lilley, Eds., Berlin-Heidelberg: Springer-Verlag; pp. 90-109), and the FLP/FRT system from the Saccharomyces cerevsiae 2μ circle plasmid (Broach et al. (1982) Cell 29: 227-234). Other examples include the pSF 1 recombinase of Zygosaccharaomyces rouxii. Members of a second family of site-specific recombinases, the resolvase family (e.g., gamma delta, Tn3 resolvase, Hin, Gin, and Cin) are also suitable for use in this invention. Although members of this highly related family of recombinases are typically constrained to intramolecular reactions (e.g., inversions and excisions) and can require host-encoded factors, mutants have been isolated that relieve some of the requirements for host factors (Maeser and Kahlnann (1991) Mol. Gen. Genet. 230: 170-176), as well as some of the constraints of intramolecular recombination.

Recombination is typically achieved by passing the library through a host cell that expresses the recombinase system. However, recombination may also be performed in vitro.

While Cre and Int are described in detail for reasons of example, many related recombinase systems exist and their application to the described invention is also provided according to the present invention. As indicated above, the integrase family of site-specific recombinases can be used to provide alternative recombination proteins and recombination sites for the present invention, as site-specific recombination proteins encoded by bacteriophage lambda, phi 80, P22, P2,186, P4 and PI. While group of proteins exhibits an unexpectedly large diversity of sequences all of these recombinases can be aligned in their C-terminal halves were also obtained in Bacillus subtilis and B. thuringiensis (see, e. g., Mahillon et al., (1994) Genetica 93: 13-26; Campbell (1992) J. Bacteriol. 7495-7499.

Often, a Cre recombination system is used in the invention. Cre, a protein from bacteriophage P1 (Abremski & Hoess, J. Biol. Chem. 259:1509-1514, 1984) catalyzes the recombination between lox P sites (see, e.g., Hoess et al., Nucl. Acids Res. 14:2287, 1986). Recombination mediated by Cre is reversible. Typically recombination is performed by passing through a host that expresses Cre. However, Cre is commercially available and, if necessary, recombination can be performed in vitro.

The recombination sites for Cre recombinase are termed lox sites. One example of a lox site is loxP, which is a 34 base pair sequence comprised of two 13 base pair inverted repeats (serving as the recombinase binding sites) flanking an 8 base pair core sequence. (See, e.g., Sauer, Curr. Opin. Biotech. 5:521-527, 1994). A number of mutant lox P sites have also been described (see, eg., Hoess et al., supra). One of these loxP, 511 recombines with another loxP 411 site, but will not recombine with a loxP site.

Typically, the loxP and loxP 511 sites are used for Cre-mediated recombination. Various mutations of this sequence such as have been described in the literature (see, e. g., Mack et al., supra; Hoess et al. (1986), supra; Hoess et al. (1984) Biochem″81: 1026-29; Hoess et al. (1985) Gene, 40: 325-329; Abremski et al. (1986) J. Biolog. Chem., 261: 391-396) are also suitable. Similar mutated sequences of loxP, which are yet to be isolated also can be employed, so long as such sequences are capable of serving as recombining sites for Cre. Intracellularly expressed recombinase is typically present in sufficient concentration to adequately drive recombination in the methods of this invention. Where an exogenous recombinase protein is supplied, the amount of recombinase that drives the recombination reaction can be determined by using known assays. Specifically, a titration assay can be used to determine the appropriate amount of a purified recombinase enzyme, or the appropriate amount of an extract.

Another example of a recombination system is the lambda integrase system. Recognition sequences include the attB, attP, attL, and attR sequences which are recognized by the recombination protein, lambda Integrase. The attB site is an approximately 25 base pair sequence containing two 9 base pair core-type Int binding sites and a 7 base pair overlap region, while attP is an approximately 240 base pair sequence containing core-type Int binding sites and arm-type Int binding sites as well as sites for auxiliary proteins integration host factor (IHF), FIS and excisionase (Xis). (See Landy, Curr. Opin. Biotech. 3:699-707 (1993).

Integrase mediates the integration of the lambda genome into the E. coli chromosome. The bacteriophage λ Int recombinational proteins promote irreversible recombination between its substrate att sites as part of the formation or induction of a lysogenic state. Reversibility of the recombination reactions results from two independent pathways for integrative and excisive recombination. Each pathway uses a unique, but overlapping, set of the 15 protein binding sites that comprise att site DNAs. Cooperative and competitive interactions involving four proteins (Int, Xis, IHF and FIS) determine the direction of recombination. Integrative recombination involves the Int and IHF proteins and sites attP (240 bp) and attB (25 bp). Recombination results in the formation of two new sites: attL and attR. Excisive recombination requires Int, IHF, and Xis, and sites attL and attR to generate attP and aftB. Under certain conditions, FIS stimulates excisive recombination. In addition to these normal reactions, it should be appreciated that attP and attB, when placed on the same molecule, can promote excisive recombination to generate two excision products, one with attL and one with attR. Similarly, intermolecular recombination between molecules containing attL and attR, in the presence of Int, IHF and Xis, can result in integrative recombination and the generation attP and attB. Hence, by flanking DNA segments with appropriate combinations of engineered att sites, in the presence of the appropriate recombination proteins, one can direct excisive or integrative recombination, as reverse reactions of each other.

Each of the att sites contains a 15 bp core sequence; individual sequence elements of functional significance lie within, outside, and across the boundaries of this common core (Landy (1989) Ann. Rev. Biochem. 58: 913). Efficient recombination between the various att sites requires that the sequence of the central common region be identical between the recombining partners, however, the exact sequence is now found to be modifiable. Consequently, derivatives of the att site with changes within the core are now discovered to recombine as least as efficiently as the native core sequences.

Integrase acts to recombine the attP site on bacteriophage lambda (about 240 bp) with the attB site on the E. coli genome (about 25 bp) (Weisberg and Landy (1983) In Lambda II, p 211, Cold Spring Harbor Laboratory)), to produce the integrated lambda genome flanked by attL (about 100 bp) and attR (about 160 bp) sites. In the absence of Xis (see below), this reaction is essentially irreversible. The integration reaction mediated by integrase and IHF also works in vitro, with simple buffer containing spermidine. Integrase can be obtained as described by Nash (1983) Meth. Enzym., 100: 210-216. IHF can be obtained as described by Filutowicz et al. (1994) Gene 147: 149-150. In the presence of the. lambda. protein Xis (excise) integrase catalyzes the reaction of attR and attL to form attP and attB, i. e., it promotes the reverse of the reaction described above. This reaction can also be applied in the present invention.

Accordingly, the present invention also provides engineered recombination sites that overcome these problems. For example, att sites can be engineered to have one or multiple mutations to enhance specificity or efficiency of the recombination reaction and the properties of product DNAs (e. g., att1, att2, and att3 sites); to decrease reverse reaction (e. g., removing PI and HI from attB). The testing of these mutants determines which mutants yield sufficient recombinational activity to be suitable for recombination according to the present invention.

Mutations can be introduced into recombination sites for enhancing site specific recombination. Such mutations include, but are not limited to: recombination sites without translation stop codons that allow fusion proteins to be encoded; recombination sites recognized by the same proteins but differing in base sequence such that they react largely or exclusively with their homologous partners allow multiple reactions to be contemplated.

There are well known procedures for introducing specific mutations into nucleic acid sequences. A number of these are described in Ausubel et al. (1989-1996) Current Protocols in Molecular Biology, Wiley Interscience, New York. Mutations can be designed into oligonucleotides, which can be used to modify existing cloned sequences, or in amplification reactions. Random mutagenesis can also be employed if appropriate selection methods are available to isolate the desired mutant DNA or RNA. The presence of the desired mutations can be confirmed by sequencing the nucleic acid by well known methods.

A number of methods can be used to engineer a core region of a given recombination site to provide mutated sites suitable for use in the present invention. These include, but are not limited to mutation of the desired core sequence (e.g. via site-specific mutagenesis, error prone PCR, chemical mutagenesis, etc) or by recombination of two parental DNA sequences by site-specific (e.g. attL and attR to give attB) or other (e. g. homologous) recombination mechanisms.

The functionality of the mutant recombination sites can be demonstrated in ways that depend on the particular characteristic that is desired. For example, the lack of translation stop codons in a recombination site can be demonstrated by expressing the appropriate fusion proteins. Specificity of recombination between homologous partners can be demonstrated by introducing the appropriate molecules into in vitro reactions, and assaying for recombination products as described herein or known in the art. Other desired mutations in recombination sites might include the presence or absence of restriction sites, translation or transcription start signals, protein binding sites, and other known functionalities of nucleic acid base sequences.

Reporter Sequences and Additional Fusion Protein Sequences

The reporter protein can comprises the complete polypeptide sequence or a subsequence that comprises the domain or domains that have reporter activity. The nucleic acid sequences encoding the reporter are located downstream of the cloning site and are out of frame with the cloning site in the absence of a cloned fragment. Accordingly, the reporter activity is present when an open reading frame that is present in the fragment subcloned into the cloning site restores the reading frame and allows read through translation to generate a fusion protein.

Reporter activity refers to any of a variety of detectable phenotypes, e.g., screenable or selectable phenotypes, such as resistance to antibiotics, color, fluorescence, growth in the presence or absence of particular substrates, and the like. Often, genes encoding proteins that provide antibiotic resistance, such as β-lactamase, are used in the systems of the invention. Other antibiotic resistance enzymes include, but are not limited to, aminoglycoside phosphotransferases, such as neomycin phosphotransferase, chloramphenicol acetyl transferase, and the tetracycline resistance protein.

Other proteins that directly elicit a visible phenotypic change such as a color change or fluorescence emission can also be used as reporter molecules. After detection of the reporter activity, colonies are selected and analyzed for the presence of an open reading frame. Example of such proteins include β-galactosidase, green fluorescent protein, red fluorescent protein, or other related fluorescent proteins.

In some embodiments, typically those in which an additional nucleic acid sequence encoding a second, additional protein is included downstream of the reporter, the nucleic acid sequence encoding the reporter protein is typically removed from the construct after selection or screening for the presence of reporter activity. This permits the generation of a fusion protein comprising the open reading frame fused to the second protein. The second protein can be any desired protein, but is typically a component of a system to assess the ability of the open reading frame to bind to other molecules; or a detectable protein, such as a polypeptide tag to be used in purification.

Components of a screening system that may be included as additional polypeptides include, e.g., display proteins or members of a two hybrid system. Display proteins include any number of virus or bacterial display proteins. Often viral coat proteins are used as display protein. These include proteins from filamentous phage, lambda phage, T7 phage, T4 phage, T2 phage and the like. Other display proteins can be, for example, from bacteria, e.g., E. coli flagellin, or from yeast, e.g., pYD1 system, or mammalian display systems.

A nucleic acid sequence encoding a member of a screening systems such as a two hybrid system may also be included as the additional sequence in the vectors and libraries of the invention. Two hybrid systems and comparable screening systems are well known in the art (see, e.g., Sambrook & Russell, supra). For example, a nucleic acid sequence encoding an activation domain, such as a GAL4 activation, may be included in a vector of the invention to generate a fusion protein comprising an open reading frame joined to the activation domain.

As appreciated by one of skill in the art, nucleic acid sequences encoding any other polypeptide, such as a selectable polypeptide, may also be incorporated into the vectors and libraries of the invention to produce fusion proteins comprising the polypeptide. For example, seqeunces encoding a selectable polypeptides such as a peptide purification tag, e.g., His, V5, and the like, can be included to produce a tag to isolate the fusion proteins.

Expression Systems and Host Cells

There are many expression systems for producing the fusion polypeptides comprising sequences encoded by an open reading frame. These systems are well know to those of ordinary skill in the art. (See, e.g., GENE EXPRESSION SYSTEMS, Fernandez and Hoeffler, Eds. Academic Press, 1999; Sambrook and Russell, supra; Ausubel, supra.) Typically, the polynucleotide that encodes the sequences to be expressed is placed under the control of a promoter that is functional in the desired host cell. A variety of promoters are available, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, the nucleic acids that encode the joined polypeptides are incorporated for expression in a desired host cell.

Fusion polypeptides can be expressed in a variety of host cells. Often bacterial hosts and expression systems, in particular gram negative bacteria such as E. coli, are employed, but other systems such as yeast, insect, fungal, plant, avian, or mammalian expression systems can also be used. Expression control sequences that are suitable for use in a particular host cell are well known to those of skill in the art. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) 8: 4057), the tac promoter (DeBoer, et al., Proc. Natl. Acad. Sci. U.S.A. (1983) 80:21-25) the hybrid trp-lac promoter; the bacteriophage T7 promoter, T3 promoter, SP6 promoter, and the lambda-derived P_{L} promoter and N-gene ribosome binding site (Shimatake et al., Nature (1981) 292: 128).

Phagemid vectors can also be employed, for example, for constructing a library of fragments to test for the presence of open reading frames. Such vectors include the origin of DNA replication from the genome of a single-stranded filamentous bacteriophage, e.g., M13 or fl. A phagemid can be used in the same way as an orthodox plasmid vector, but can also be used to produce filamentous bacteriophage particle that contain single-stranded copies of cloned segments of DNA.

Any available promoter that functions in prokaryotes can be used, although the particular promoter system can be selected for optimal expression as further addressed below. Standard bacterial expression vectors include plasmids such as pBR322-based plasmids, e.g., pBLUESCRIPT™, pSKF, pET23D, λ-phage derived vectors, and fusion expression systems such as GST and LacZ. Epitope tags can also be added to recombinant proteins to provide convenient methods of isolation, e.g., c-myc, HA-tag, 6-His tag, maltose binding protein, VSV-G tag, anti-DYKDDDDK tag, or any such tag, a large number of which are well known to those of skill in the art.

For expression of fusion polypeptides in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli. These and other suitable bacterial promoters are well known in the art and are described, e.g., in Sambrook et al. and Ausubel et al. Bacterial expression systems for expressing the proteins of the invention are available in, e.g., E. coli, Bacillus sp., and Salmonella (Palva et al., Gene 22:229-235 (1983); Mosbach et al., Nature 302:543-545 (1983). Kits for such expression systems are commercially available.

Either constitutive or regulated promoters can be used in the present invention. Regulated promoters can be advantageous because the host cells can be grown to high densities before expression of the fusion polypeptides is induced. High level expression of heterologous proteins slows cell growth in some situations. An inducible promoter is a promoter that directs expression of a gene where the level of expression is alterable by environmental or developmental factors such as, for example, temperature, pH, anaerobic or aerobic conditions, light, transcription factors and chemicals.

For E. coli and other bacterial host cells, inducible promoters are known to those of skill in the art. These include, for example, the lac promoter, the bacteriophage lambda P_{L} promoter, the hybrid trp-lac promoter (Amann et al. (1983) Gene 25: 167; de Boer et al. (1983) Proc. Nat'l. Acad. Sci. USA 80: 21), and the bacteriophage T7 promoter (Studier et al. (1986) J. Mol. Biol.; Tabor et al. (1985) Proc. Nat'l. Acad. Sci. USA 82: 1074-8). These promoters and their use are discussed in Sambrook et al., supra.

In some applications, eukaryotic expression systems can be used in practicing the methods of the invention. For example, yeast expression systems are well known in the art and can also be used. In yeast, vectors include Yeast Integrating plasmids (e.g., YIp5) and Yeast Replicating plasmids (the YRp series plasmids) and pGPD-2.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells. Inducible promoters for eukaryotic organisms are also well known to those of skill in the art. These include, for example, the metallothionein promoter, the heat shock promoter, as well as many others.

Translational coupling may be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See Squires, et. al (1988), J. Biol. Chem. 263: 16297-16302.

The polypeptide fusions can be expressed intracellularly, or can be secreted from the cell or into the periplasmic space. The expression construct can therefore include sequence, e.g., a leader or signal sequence to allow secretion of the expressed protein.

To facilitate purification of expressed polypeptides, the nucleic acids that encode the fusion polypeptides can also include a coding sequence for an epitope or “tag” for which an affinity binding reagent is available. Examples of suitable epitopes include the myc and V-5 reporter genes; expression vectors useful for recombinant production of fusion polypeptides having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the fusion proteins of the invention, and corresponding detection systems are known to those of skill in the art, and several are commercially available (e.g., FLAG″ (Kodak, Rochester N.Y.). Another example of a suitable tag is a polyhistidine sequence, which is capable of binding to metal chelate affinity ligands. Typically, six adjacent histidines are used, although one can use more or less than six. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, NY; commercially available from Qiagen (Santa Clarita, Calif.)).

Display Libraries

The systems of the invention can be used in a number of applications. Often, the vectors are used to construct phage display libraries that are largely comprised by fragments that are open reading frames. In this embodiment, the vectors include a nucleic acid sequence encoding a display protein downstream of the reporter sequence. Removal of the reporter sequence results in an open reading frame-display protein fusion. The open reading frame can then be displayed on the surface of a particle, e.g., a virus or cell and screened for the ability to interact with other molecules, e.g., a library of antibodies.

Construction of phage display libraries exploits the bacteriophage's ability to display peptides and proteins on their surfaces, i.e., on their capsids. Often, filamentous phage such as M13 or fl are used. Filamentous phage contain single-stranded DNA surrounded by multiple copies of genes encoding major and minor coat proteins, e.g., pIII. Coat proteins are displayed on the capsid's outer surface. DNA sequences inserted in-frame with capsid protein genes are co-translated to generate fusion proteins or protein fragments displayed on the phage surface. Peptide phage libraries thus can display peptides representative of the diversity of the inserted genomic sequences. Significantly, these epitopes can be displayed in “natural” folded conformations. The peptides expressed on phage display libraries can then bind target molecules, i.e., they can specifically interact with binding partner molecules such as antibodies (Petersen (1995) Mol. Gen. Genet. 249:425-31), cell surface receptors (Kay (1993) Gene 128:59-65), and extracellular and intracellular proteins (Gram (1993) J. Immunol. Methods 161:169-76).

The concept of using filamentous phages, such as M13 or fd, for displaying peptides on phage capsid surfaces was first introduced by Smith (1985) Science 228:1315-1317. Peptides have been displayed on phage surfaces to identify many potential ligands (see, e.g., Cwirla (1990) Proc. Natl. Acad. Sci. USA 87:6378-6382). There are numerous systems and methods for generating phage display libraries described in the scientific and patent literature, see, e.g., Sambrook and Russell, Molecule Cloning: A Laboratory Manual, 3rd edition, Cold Spring Harbor Laboratory Press, Chapter 18, 2001; “Phage Display of Peptides and Proteins: A Laboratory Manual, Academic Press, San Diego, 1996; Crameri (1994) Eur. J. Biochem. 226:53-58; de Kruif (1995) Proc. Natl. Acad. Sci. USA 92:3938-42; McGregor (1996) Mol. Biotechnol. 6:155-162; Jacobsson (1996) Biotechniques 20:1070-1076; Jespers (1996) Gene 173:179-181; Jacobsson (1997) Microbiol Res. 152:121-128; Fack (1997) J. Immunol. Methods 206:43-52; Rossenu (1997) J. Protein Chem. 16:499-503; Katz (1997) Annu. Rev. Biophys. Biomol. Struct. 26:27-45; Rader (1997) Curr. Opin. Biotechnol. 8:503-508; Griffiths (1998) Curr. Opin. Biotechnol. 9:102-108.

Typically, exogenous nucleic acid to be displayed are inserted into a coat protein gene, e.g. gene III or gene VIII of the phage. The resultant fusion proteins are displayed on the surface of the capsid. Protein VIII is present in approximately 2700 copies per phage, compared to 3 to 5 copies for protein III (Jacobsson (1996), supra). Multivalent expression vectors, such as phagemids, can be used for manipulation of exogenous genomic or antibody encoding inserts and production of phage particles in bacteria (see, e.g., Felici (1991) J. Mol. Biol. 222:301-310).

Phagemid vectors are often employed for constructing the phage library. These vectors include the origin of DNA replication from the genome of a single-stranded filamentous bacteriophage, e.g., M13 or fl. A phagemid can be used in the same way as an orthodox plasmid vector, but can also be used to produce filamentous bacteriophage particle that contain single-stranded copies of cloned segments of DNA.

Other phage can also be used. For example, T7 vectors can be employed in which the displayed product on the mature phage particle is released by cell lysis.

Another useful methodology is selectively infective phage (SIP) technology. which provides for the in vivo selection of interacting protein-ligand pairs. A “selectively infective phage” consists of two independent components. A recombinant filamentous phage particle is made non-infective by replacing its N-terminal domains of gene 3 protein (g3p) with a ligand-binding protein. For example, the genomic nucleic acid to be mapped can be inserted such that it will be expressed as this ligand-binding protein. The second component is an “adapter” molecule in which the ligand is linked to those N-terminal domains of g3p which are missing from the phage particle. Infectivity is restored when the displayed protein (e.g., a “binding site”) binds to the epitope ligand. This interaction attaches the missing N-terminal domains of g3p to the epitope phage display particle. Phage propagation becomes strictly dependent on the protein-ligand interaction. See, e.g., Spada (1997) J. Biol. Chem. 378:445-456; Pedrazzi (1997) FEBS Lett. 415:289-293; Hennecke (1998) Protein Eng. 11:405-410.

In addition to phage epitope display libraries, analogous epitope display libraries can also be used. For example, the methods of the invention can also use yeast surface displayed epitope libraries (see, e.g., Boder, Nat. Biotechnol. 15:553-557, 1997), which can be constructed using such vectors as the pYD1 yeast expression vector. Other potential display systems include mammalian display vectors and E. coli libraries. For example, the E. coli flagellin protein can be used to display sequences encoded by open reading frames.

In constructing a library, the open reading frame can also be fused to a scaffold protein. A scaffolded peptide refers to a peptide, typically of up to about 20 amino acids in length, that is inserted into a natural protein at a location known to accept such insertions without interfering with the folding or native configuration of the protein. Many proteins may serve as scaffolds for libraries. Examples of proteins that have been used as scaffolds include, but are not limited to, thioredoxin (or other thioredoxin-like proteins), nucleases (e.g., RNase A), proteases (e.g., trypsin), protease inhibitors (e.g., bovine pancreatic trypsin inhibitor), antibodies or structurally-rigid fragments thereof, and other domains of the immunoglobulin superfamily.

As appreciated by one of skill in the art, the vectors of the invention can be used to generate libraries other than display libraries. For example, a nucleic acid sequence located downstream of the reporter can be a component of a two-hybrid system such as an activation domain. Upon removal of the reporter sequence, a fusion of the open reading frame and activation domain is created. A library of open reading frames thus created can then be screened for the ability to interact with another sequence that is fused to the second component of the two hybrid system, e.g., a DNA binding domain

Thus, the vectors and methods of the invention can be used to generate diverse libraries that are greatly enriched for open reading frames.

Although the invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

All publications, patents, and patent applications cited in this specification are herein incorporated by reference as if each individual publication or patent application were specifically and individually indicated to be incorporated by reference.

### EXAMPLES

### Example 1

Design and Testing of pPAOLA2

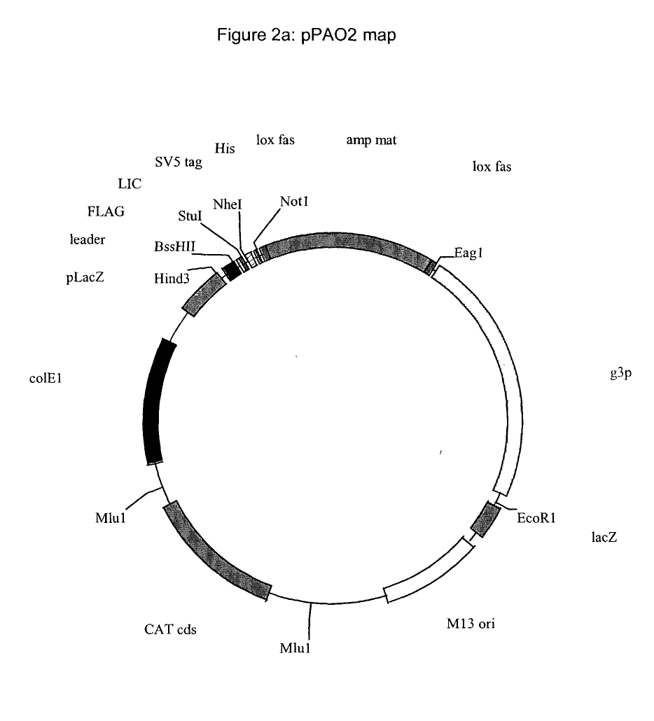

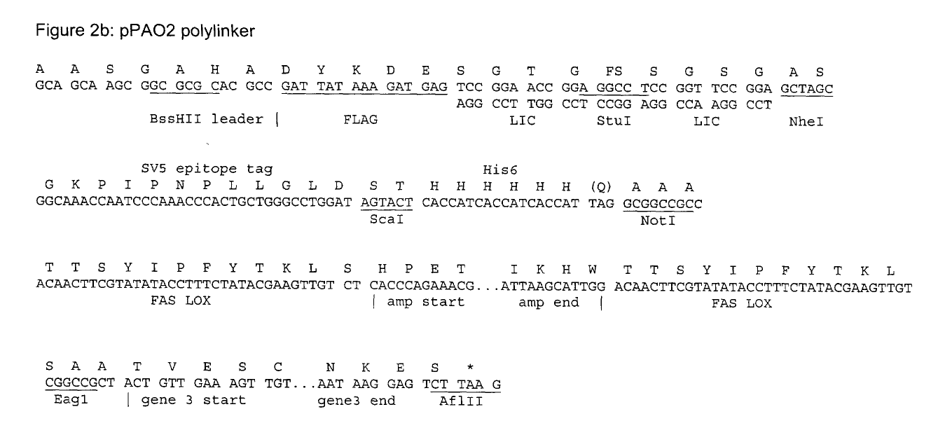

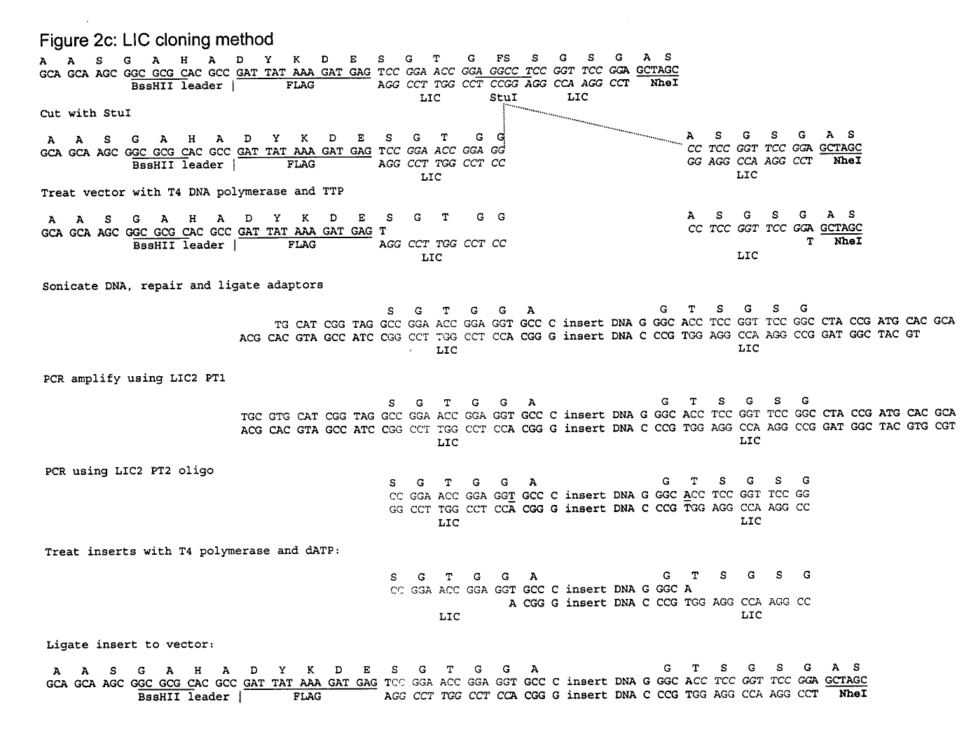

The essential features of pPAO2 are illustrated in FIG. 2. Blunt ended fragments could be cloned using either a blunt site (StuI) or Ligation Independent Cloning (LIC). The vector is designed to take advantage of the ligation-independent cloning strategy, in which no restriction enzymes are used, to avoid potential bias. Briefly, the vector contains a StuI site surrounded by two 12 nucleotide-long palindromic sequences lacking dTMP. After cutting with StuI, the two blunt ends generated are degraded by the 3′−>5′ exonuclease activity of T4 DNA polymerase in the presence of T4 DNA polymerase and dTTP. As a result of the sequence design, the exonuclease stops at nucleotide 12, which is dTMP, thus creating short cohesive ends required for LIC adaptor mediated cloning. These cloning sites are upstream of a beta lactamase gene flanked by two lox recombination signals. In addition, the vector also contains tags for two commonly used monoclonals, FLAG and SV5, as well as a His tag for purification by immobilized metal affinity chromatography, and chloramphenicol resistance in the backbone of the plasmid. The polylinker is out of frame with respect to the ampicillin (indicated by FS in FIG. 2), and can only be returned into frame if DNA containing an open reading frame with 3n+2 nucleotides is correctly cloned.

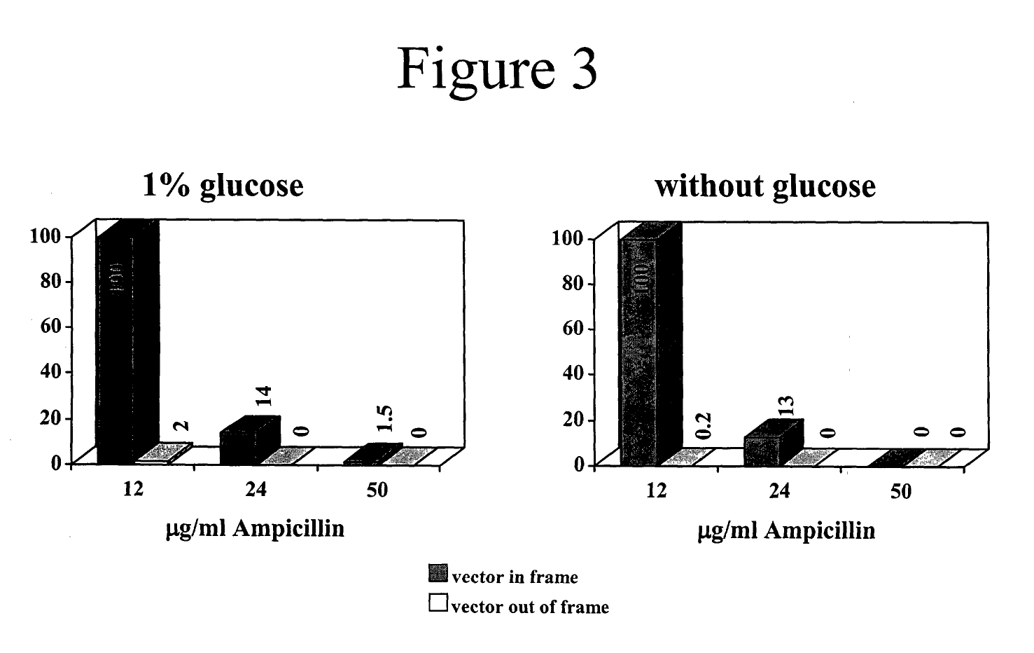

In order to examine the efficiency of the selection for open reading frames, DNA encoding either a single chain Fv fragment (D1.3), or an out-of-frame derivative, were cloned between the BssHII and NheI sites. As shown in FIG. 3, by using an ampicillin concentration of 12 μg/ml in the absence of glucose, 100% of the in-frame clones survive, whereas only 0.2% of the out-of-frame clones survive. Increasing the concentration of ampicillin to 25 μg/ml reduced the percentage of in-frame clones surviving by 85%, and eliminated all out-of-frame clones, while the addition of glucose (which should inhibit transcription from the lac promoter) allowed more out-of-frame clones to survive at the lowest ampicillin concentration tested. On the basis of these results, 12 μg/ml ampicillin was used for all subsequent experiments.

After selection on ampicillin, bacteria were harvested and phagemid were prepared. The efficiency of the recombination-mediated removal of the β lactamase gene was tested by infecting these phagemid into BS 1365, an F′ bacteria constitutively expressing cre recombinase, and allowing recombination to occur overnight at 30° C. Phagemid were prepared from these bacteria, reinfected into DH5αF′ and plated out onto chloramphenicol (24 μg/ml) or ampicillin (12 μg/ml) plates. The number of colonies growing on ampicillin plates was always less than 1% of those growing on chloramphenicol. To further confirm these results, 100 colonies were picked from the chloramphenicol plate and replated on either ampicillin (12 μ/ml) or chloramphenicol (24 μg/ml) plates. All colonies grew on the chloramphenicol plates, and none grew on the ampicillin plates, suggesting that the β lactamase gene had been efficiently removed by cre recombinase. This was also confirmed by PCR, which showed the removal of the B lactamase gene in the 20 clones tested (data not shown).

Although, the ampicillin gene was removed by recombination, the lox recombination signal remains as a translated “linker” between the displayed protein and p3. In order to find out whether this affected the efficiency of display, three different D1.3 phagemid were tested for display efficiency: pDAN5-D1.3 (a standard phage antibody vector 48) and pPAO2-D1.3 before or after the removal of β lactamase by recombination. The ability of the phage to bind to lysozyme (the antigen recognized by D 1.3) was tested by ELISA. As shown in Table 1, the ELISA signals given by pDAN5-D1.3 and pPAO2-D1.3 after recombination were similar, indicating that the translated lox linker did not inadvertanty affect display. The presence of β-lactamase between D1.3 and p3, however, had a notable effect on display efficiency.

TABLE 1

ELISA signals

| construct | Lysozyme | BSA |
|---|---|---|
| pDAN5 D1.3 | 0.934 ± 0.120 | 10.090 ± 0.021 |
| pPAO1 D1.3 before cre | 0.126 ± 0.013 | 10.086 ± 0.007 |
| pPAO1 D1.3 after cre | 0.878 ± 0.080 | 10.102 ± 0.038 |

### Example 2

pPAO2 can Select Open Reading Frames

In order to evaluate the efficiency of pPAO2 in filtering out open reading frames from random DNA, a library of fragments from the tissue transglutaminase (tTG)-encoding plasmid, pET28b-tTG 50, was prepared. This plasmid can be considered to be a mini-genome, containing four known genes accounting for approximately 50% of the DNA, and containing an additional 62 open reading frames greater than 50 amino acids in length. The library was made by digesting the plasmid with DNAse to fragments of 100-300 bp, repairing them with Pfu DNA polymerase, and ligating to LIC adaptors to create the 12 nucleotides single stranded overhang complementary to the pPAO2 cohesive ends created using T4 DNA polymerase and dATP. The scheme for this cloning method is shown in FIG. 2. A small aliquot of the library was plated on chloramphenicol and ampicillin plates. The number of colonies obtained on the chloramphenicol plates was approximately eighty-fold greater than the number of colonies obtained on the ampicillin plates (Table 2), indicating that strong selection had occurred. The library obtained after plating on ampicillin plates contained 7000 colonies. The β lactamase gene was removed from the ampicillin-resistant colonies by infecting phagemid made from the colonies into BS1365 (which expresses cre recombinase constitutively), and reinfecting phagemid produced by the bacteria into DH5αF′.

TABLE 2

| Number of colonies | | Ratio |
|---|---|---|
| Chloramphenicol | Ampicillin | chloramphenicol/ |
| (24 μg/ml) | (12 μg/ml) | ampicillin |
| 32000 | 400 | 80:1 |

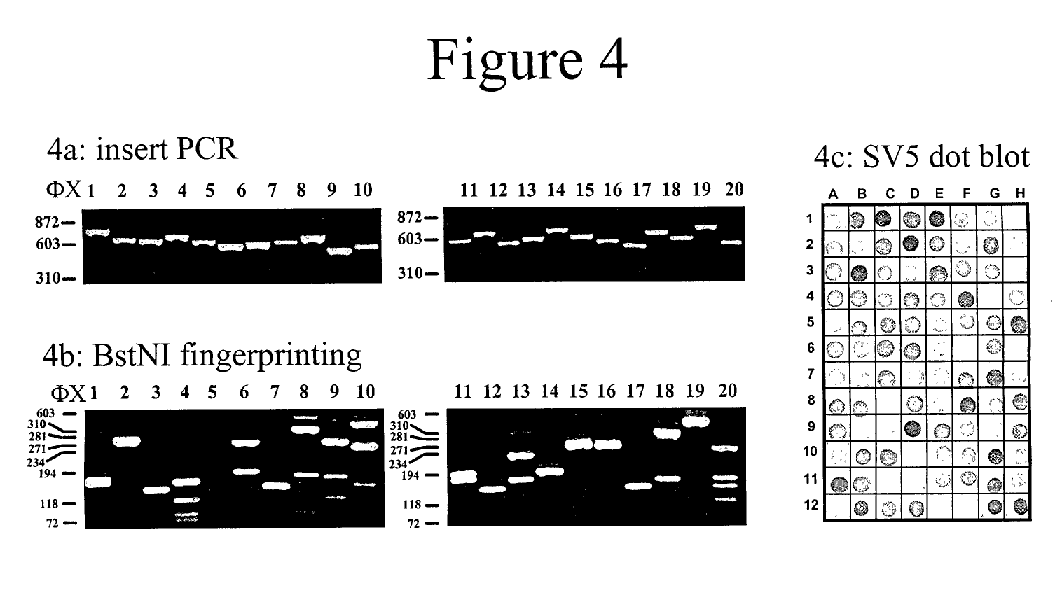

PCR amplification of a number of different inserts was then conducted to characterize the library from which the β-lactamase had been removed (FIG. 4a), and the diversity of the inserts was examined by BstNI fingerprinting (FIG. 4b). The inserts were of different sizes and showed different digestion patterns, indicating that no single clone dominated. 96 colonies were plated out on a chloramphenicol IPTG plate and examined in a dot blot for expression of the SV5 tag, which is positioned between the cloned insert, and p3 and will only be in frame if the cloned insert is in frame (FIG. 2). As shown in FIG. 4c, at least 80 colonies showed SV5 binding (different signals are attributable to differences in leakage of the fusion protein out of the bacteria), indicating that a large proportion of the library consisted of open reading frames. This was confirmed by sequencing 43 random colonies (Table 3a) all of which were found to be in frame.

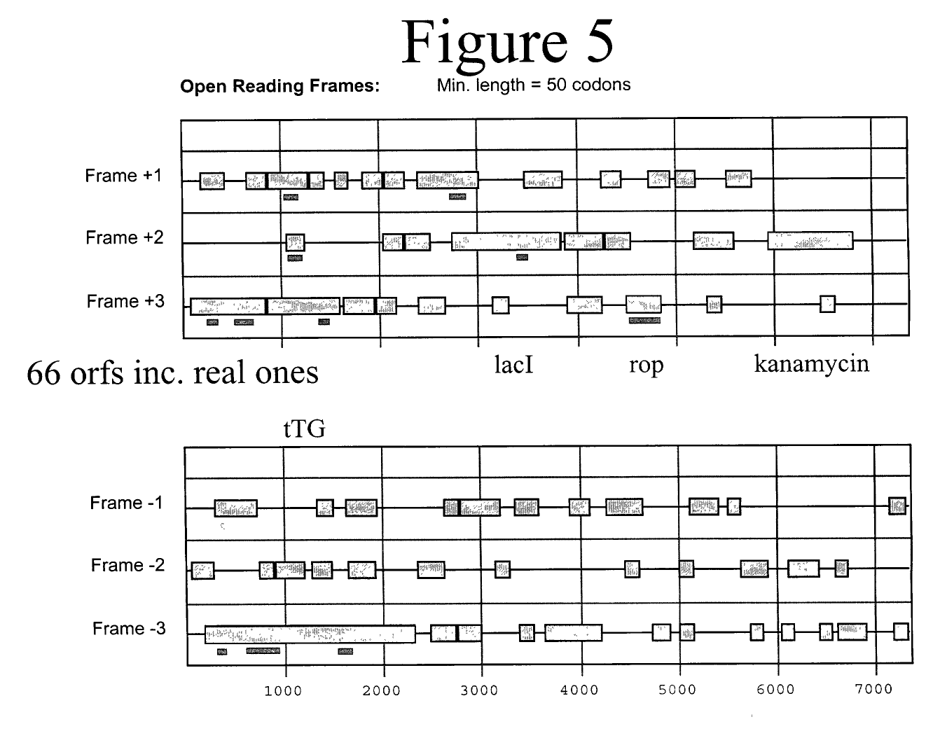

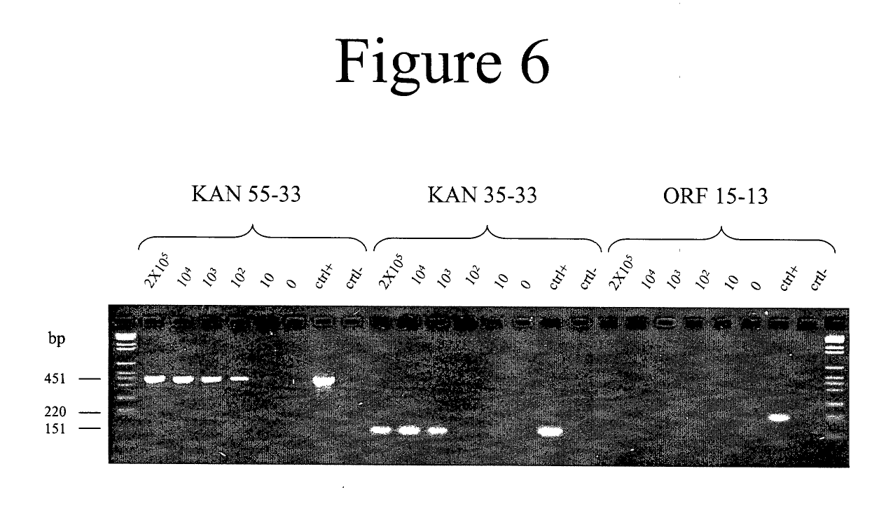

The ORFs from which the clones were derived is indicated in the table, and represented graphically in FIG. 5, in which all open reading frames greater than 50 amino acids (150bp) are shown. Those which were found in the random sequencing are indicated by the thick red lines. Of the four functional ORFs in pET28b-tTG, three (lacI, tTG and rop) were represented and comprised 84% of all the clones present. The kanamycin resistance gene was not found among the 43 sequenced clones. In order to determine whether this open reading frame was found in the tTG ORF library, three nested primers amplifying fragments of 451 or 154 bp were designed. As shown in FIG. 6, both the 451 and the 154 fragments were found when at least 100 templates were present in the PCR reaction, whereas a randomly picked ORF of no biological significance could not be detected in the whole library.

TABLE 3a
random sequenced clones

| Clone | Number | Origin | Start | End | ORF | length |
|---|---|---|---|---|---|---|
| A2 | 1 | E. coli | — | — | | 161 |
| G3 | 1 | pET | 2693 | 2870 | 2389-3024 | 178 |
| F9 | 1 | pET | 3531 | 3591 | LacI | 61 |
| | | | | | (2741-3856) | |
| C5 | 2 | pET | 4528 | 4837 | Rop | 310 |
| | | | | | (4497-4856) | |
| C3 | 1 | pET | 4594 | 4768 | rop | 175 |
| B6 | 1 | pET | 4603 | 4784 | rop | 187 |
| B3 | 2 | pET | 4618 | 4780 | rop | 163 |
| C9 | 2 | pET | 4627 | 4789 | rop | 163 |
| C11 | 1 | pET | 4630 | 4762 | rop | 133 |
| G1 | 1 | pET | 4630 | 4765 | rop | 136 |
| E12 | 1 | pET | 4630 | 4768 | rop | 139 |
| D3 | 1 | pET | 4630 | 4783 | rop | 154 |
| G11 | 2 | pET | 4630 | 4837 | rop | 208 |
| B4 | 2 | pET | 4630 | 4849 | rop | 220 |
| D1 | 1 | pET | 4636 | 4855 | rop | 220 |
| H1 | 1 | pET | 4636 | 4837 | rop | 202 |
| F3 | 1 | pET | 4639 | 4780 | rop | 142 |
| A1 | 1 | pET | 4651 | 4765 | rop | 115 |
| A3 | 1 | pET | 4551 | 4768 | rop | 118 |
| A7 | 5 | pET | 4752 | 4804 | rop | 154 |
| H8 | 1 | pET | 4654 | 4804 | rop | 151 |
| D12 | 1 | pET | 4669 | 4789 | rop | 121 |
| E6 | 1 | tTG | 1600 | 1483 | tTG | 118 |
| | | | | | (2330-198) | |
| A5 | 1 | tTG | 928 | 745 | tTG | 184 |
| E10 | 1 | tTG | 874 | 733 | tTG | 142 |
| B3 | 2 | tTG | 724 | 612 | tTG | 113 |
| D4 | 1 | tTG | 265 | 385 | tTG | 121 |
| B2 | 1 | tTG- | 1089 | 1266 | 1067-1264 | 178 |
| D8 | 1 | tTG- | 1525 | 1600 | 855-1613 | 76 |
| F2 | 1 | tTG- | 1039 | 1222 | 877-1302 | 184 |
| B11 | 2 | tTG- | 568 | 730 | 7221-854 | 163 |
| D2 | 1 | tTG- | 262 | 379 | 7221-854 | 118 |

TABLE 3b
Clones selected* or positively screened° on mAb CUB (epitope 860-768)

| Clone | Number | Origin | Start | End | ORF | length |
|---|---|---|---|---|---|---|
| D9* | 15 | tTG | 982 | 739 | tTG | 244 |
| E4° | 1 | tTG | 991 | 766 | tTG | 226 |
| F6° | 1 | tTG | 883 | 700 | tTG | 184 |
| D12 | 1 | tTG | 412 | 262 | tTG | 151 |
| A4 | 1 | pET | 4651 | 4804 | rop | 154 |

### Example 3

Selecting and Screening Using the pPAOA2 Library

In order to determine whether this approach to selecting open reading frames provides libraries of open reading frames suitable for subsequent selection or screening experiments, the library was selected on a monoclonal antibody, CUB that recognizes a linear epitope (860-768 in the plasmid) in transglutaminase. After a single round of selection, 15 identical clones were identified which overlapped the known epitope. As an alternative to selection, 96 randomly picked clones were tested by dot blot for binding to CUB, and four further clones were found to be positive. Two of these also corresponded to the known epitope, while the remaining clones, which had much weaker signals, corresponded to irrelevent sequences.

### MATERIALS AND METHODS

### Bacterial Strains

The bacterial strains used in the current examples were: Dh5αF′ (Gibco BRL): F′/endA1 hsd17 (r_{K}⁻ m_{K}⁺) supE44 thi-1 recA1 gyrA (Nal^{r}) relA1 Δ (lacZYA-argF) U169 deoR (F80dlacD(lacZ)M15): and BS1365: BS591 F′ Kan (BS591: recA1 endA1 gyrA96 thi-1 D lacU169 supE44 hsdR17[lamda1 mm434 nin5 X1-cre].

### Construction of Plasmid

The phagemid pPAOLA2 (FIG. 2) is a derivative of pDAN5⁴⁸ specifically modified to exploit the ligation-independent cloning method⁴⁹. A new polylinker sequence was generated by PCR using primers LIC2 Vector For (5′TTG CCG CTA GCT CCG GAA CCG GAG GCC TCC GGT TCC GGA CTC ATC TTT ATA ATC GGC ATG CGC GCC GCT TGC TGC 3′) and M13 rev seq (5′AGC GGA TAA CAA TTT CAC ACA 3′) and pDAN5 as the template. The PCR product was cloned into pDAN5 as a HindIII-NheI fragment.

### Library Construction

Fifteen μg pET28-htTG 50 was digested into random fragments by adding 0.1U of DNAseI in the presence of 50 mM Tris-HCl, 10 mM MnCl2, pH 7.5 for 1-2 minutes at 15° C., and repaired by the addition of 5U of Pfu DNA polymerase. Blunt-ended fragments of 100-300 bp were purified by electrophoresis in a 2% agarose gel and recovered from the gel using the Qiaquick Gel Extraction kit (Qiagen, Germany). These DNA fragments were ligated to LIC linkers (oligo sense 5′ TGC ATC GGT AGG CCG GAA CCG GAG GTG CCC 3′; oligo antisense 5′ GGG CAC CTC CGG TTC CGG CCT ACC GAT GCA CGC A 3′) in a reaction mixture containing LIC Adaptors (20 μM), please provide amount DNA fragments, 1×T4 DNA ligase buffer, and T4 DNA ligase overnight at 15° C. Unligated adaptors were removed using a 1 mL Sephacryl S-400 HR spin column (Pharmacia) as recommended by the supplier. The fragments with ligated adaptors were PCR amplified using primer LIC2PT1 (5′ TGC GTG CAT CGG TAG 3′) and primer LIC2PT2 (5′ CCG GAA CCG GAG G 3′) before cloning. To create the single-stranded LIC tails in the plasmid, StuI-digested vector and adaptor-ligated inserts were treated with 2 units of T4 DNA polymerase in the presence of dTTP (0.5 mM) and dATP (0.5 mM), respectively. After incubation for 20 min at 37° C. the mixtures were heat-inactivated and purified using the Qiaquick PCR Purification kit (Qiagen, Germany). For the large scale ligation reaction, 15 μg of T4 DNA polymerase-treated vector was combined with 3 μg T4 DNA Polymerase-treated PCR products in a 100 μl volume. After a 1 hr incubation at room temperature, the ligation mixture was extracted with an equal volume 50:50 phenol:chloroform followed by ethanol precipitation. The resulting DNA pellet was resuspended in 20 μl of water. Each μl of ligation reaction was then used to transform 50 μl of electrocompetent DH12S cells.

### Elisa Analysis and Dot Blot

Phage ELISA was used to identify lysozyme binding D1.3 scFvs present in either pDAN5 or pPAOALA2 with or without β-lactamase.

### PCR Analysis of the Ampicillin Resistant Transformants and DNA Sequencing

To characterise the transformants from different experiments, randomly picked recombinants were analysed for inserts by PCR with primers M13 reverse 5′-CAG GAA ACA GCT ACC-3′ and (5′ end specific) and AMP 5′-TCG ATG TAA CCC ACT CGT GC-3′ (3′ end specific). Bacterial colonies were transferred into the PCR mixture by touching the colony using disposable pipette tips and pipetting up and down in the PCR mixture. Aliquots were analysed by agarose gel electrophoresis. For BstNI fingerprinting the PCR products were digested with BstNI and the different pattern of the resulting fragments were resolved on a 2% Metaphore agarose gel. DNA sequencing was carried out using Epicentre Sequiterm ExcelII Kits (Alsbyte, Mill Valley, Calif.) and analysed using specific labeled M13 reverse and AMP-specific primers. Sequences were analysed on a Li-Cor 4000L automatic sequencer (Lincoln, Nebr.).

To analyze the library for the presence of the kanamycin gene, three primers (kan 55 TGT ATG GGA AGC CCG ATG; kan 35 GCG ATC GCG TAT TTC GTC and kan 33 GAC TGA ATC CGG TGA GAA TG) were synthesised. PCR with kan 55 and 33 would be expected to result in a band of 451 bp, while amplification with kan 35 and 33 would be expected to result in a band of 154b bp, if the kan gene was present in the library. In addition, two other primers (orfl3, 5′-CACCGGCATACTCTG-3′ and orfl 5, 5′-CATGCA CCATTCCTT-3′) corresponding to the largest open reading frame (2389-3024) were synthesised. These were expected to yield a fragment of 220 bp (2596-2816).

### β-lactamase Removal by Recombination

To induce the removal of the β-lactamase gene following selection on ampicillin plates, phagemid were prepared from pooled selected clones, as previously described, and infected into BS 1365 (bacteria constitutively expressing cre) grown in 2×TY, 100 mg/ml kanamycin, 1% glucose at 37° C. to OD₅₅₀ 0.5. Recombination was allowed to proceed by shaking overnight at 30° C. The following day, bacteria were diluted 1/20 in the same medium, grown to OD₅₅₀ 0.5 at 37° C., and infected with M13K07 helper phage at an MOI of 20:1, and left without shaking for 30 minutes at 37° C. before 2h further growth. Colonies were derived from these phagemid by infection into DH5αF′. These represent preselected open reading frames, displayed on phagemid, in which β-lactamase has been removed.

What is claimed is:

1. An expression vector comprising a cloning site upstream of a nucleic acid sequence encoding a reporter protein, and a first and a second recombination site, wherein the first recombination site is situated between the cloning site and the nucleic acid sequence encoding the reporter protein, and the second recombination site is situated downstream of the nucleic acid sequence encoding the reporter protein or upstream of the cloning site;
wherein expression of an open reading frame present in a nucleic acid fragment inserted into the cloning site results in a fusion protein comprising the sequence expressed from the open reading frame and the reporter protein.

2. The expression vector of claim 1, further comprising a nucleic acid sequence encoding an additional polypeptide that is in-frame with the recombination sites, the reporter protein, and the open reading frame.

3. The expression vector of claim 2, wherein the additional polypeptide is a purification tag.

4. The expression vector of claim 2, wherein the additional polypeptide is a component of a two-hybrid system.

5. The expression vector of claim 2, wherein the additional polypeptide is a display protein.

6. The expression vector of claim 5, wherein the display protein is a virus coat protein.

7. The expression vector of claim 6, wherein the virus is selected from the group consisting of a filamentous phage, a lambda phage, or a T7 phage.

8. The expression vector of claim 7, wherein the filamentous phage is selected from the group consisting of an fd phage, an fl phage, an M13 phage, an Ike phage, or a hybrid thereof.

9. The expression vector of claim 6, wherein the coat protein is filamentous phage gene 3, filamentous phage gene 7, filamentous phage gene 8, or filamentous phage gene 6.

10. The expression vector of claim 1, wherein the fusion protein is expressed in a bacterial cell.

11. The expression vector of claim 1, wherein the fusion protein is expressed in a yeast cell.

12. The expression vector of claim 1, wherein the first and the second recombination sites are homologous lox sites, wherein recombination between the lox sites, eliminates nucleic acid sequences between the lox sites.

13. The expression vector of claim 1, wherein the first and the second recombination sites are heterologous lox sites, wherein recombination between the lox sites, transfers the nucleic acid sequences flanked by the lox sites to another plasmid.

14. The expression vector of claim 1, wherein the reporter protein is an enzyme.

15. The expression vector of claim 1, wherein the reporter protein is encoded by an antibiotic resistance gene.

16. The expression vector of claim 15, wherein the antiobiotic resistance gene encodes β-lactamase or chloramphenicol acetyltransferase.

17. The expression vector of claim 14, wherein the reporter protein is a fluorescent protein.

18. The expression vector of claim 1, wherein the nucleic acid fragment is a genomic fragment.

19. The expression vector of claim 1, wherein the nucleic acid fragment is a cDNA fragment.

20. A method of identifying an open reading frame in a nucleic acid fragment, the method comprising:
expressing a population of nucleic acid fragments in an expression vector comprising: a cloning site upstream of at nucleic acid sequence encoding a reporter and a first and a second recombination site, wherein the first recombination site is situated between the cloning site and the nucleic acid sequence encoding the reporter protein, and the second recombination site is situated either downstream of the nucleic acid sequence encoding the reporter protein or upstream of the cloning site, wherein expression of an open reading frame present in a nucleic acid fragment inserted into the cloning site results in a fusion protein comprising the sequence expressed from the open reading frame and the reporter protein; and
selecting the vector that expresses the fusion protein.

21. The method of claim 20, further comprising a step of removing the reporter sequence by homologous recombination.

22. The method of claim 20, further comprising a step of transferring the selected open reading frame to another plasmid by recombination.

23. The method of claim 20, wherein the expression vector further comprises a nucleic acid sequence encoding an additional polypeptide, wherein the nucleic acid encoding the additional polypeptide is in-frame with the recombination sites.

24. The method of claim 23, wherein the additional polypeptide is a display protein.

25. The method of claim 24, wherein the display protein is a virus coat protein.

26. The method of claim 25, wherein the virus is selected from the group consisting of a filamentous phage, a lambda phage, or a T7 phage.

27. The method of claim 26, wherein the filamentous phage is selected from the group consisting of an fd phage, an fl phage, an M13 phage, an Ike phage, or a hybrid thereof.

28. The method of claim 25, wherein the coat protein is filamentous phage gene 3, filamentous phage gene 7, filamentous gene 8, or filamentous phage gene 6.

29. The method of claim 20, wherein the fusion protein is expressed in a bacterial cell.

30. The method of claim 20, wherein the fusion protein is expressed in a yeast cell.

31. The method of claim 20, wherein the first and the second recombination sites are lox sites.

32. The method of claim 20, wherein the reporter protein is an antiobiotic resistance gene.

33. The method of claim 20, wherein the reporter protein is a fluorescent protein.

34. The method of claim 33, wherein the fluorescent protein is green fluroescent protein.

35. The method of claim 32, wherein the antibiotic resistance gene encodes for a β-lactamase or a chloramphenicol acetyltransferase.

36. The method of claim 20, wherein the nucleic acid fragment is a genomic fragment.

37. The method of claim 20, wherein the nucleic acid fragment is a cDNA fragment.

38. A library comprising members that express a population of nucleic acid fragments that encode open reading frames, wherein an open reading expressed by a member of the library is joined in-frame to a component of a binding detection system, and further, wherein sequences that are encoded by a recombination site are present between the display protein and the open reading frame.

39. The library of claim 38, wherein the component of a binding detection system is a display protein.

40. The library of claim 39, wherein the display protein is a virus coat protein.

41. The library of claim 40, wherein the virus is selected from the group consisting of a filamentous phage, a lambda phage, or a T7 phage.

42. The library of claim 41, wherein the filamentous phage is selected from the group consisting of an fd phage, an fl phage, an M13 phage, an Ike phage, or a hybrid thereof.

43. The method of claim 40, wherein the coat protein is filamentous phage gene 3, filamentous phage gene 7, filamentous gene 8, or filamentous phage gene 6.

44. A library comprising members that express a population of nucleic acid fragments that encode open reading frames, wherein an open reading present in a nucleic acid fragment is expressed as a component of a fusion protein, wherein the fusion protein further comprises a reporter protein and a display protein.

45. The library of claim 44, wherein the component of a binding detection system is a display protein.

46. The library of claim 45, wherein the display protein is a virus coat protein.

47. The library of claim 46, wherein the virus is selected from the group consisting of a filamentous phage, a lambda phage, or a T7 phage.

48. The library of claim 47, wherein the filamentous phage is selected from the group consisting of an fd phage, an fl phage, an M13 phage, an Ike phage, or a hybrid thereof.

49. The method of claim 46, wherein the coat protein is filamentous phage gene 3, filamentous phage gene 7, filamentous gene 8, or filamentous phage gene 6.
